(19) United States
(12) Patent Application Publication
(10) Pub. No.: US 2026/0102353 A1
(43) Pub. Date: Apr. 16, 2026

(54) MULTIPARTICULATE ESSENTIAL OIL COMPOSITIONS WITH CONTROLLED RELEASE IN THE STOMACH

(71) Applicant: Digest Pharma LLC, Boynton Beach, FL (US)

(72) Inventor: Syed Muzafar Shah, Boynton Beach, FL (US)

(21) Appl. No.: 18/912,227

(22) Filed: Oct. 10, 2024

(51) Int. Cl. A61K 9/20; A61K 36/23; A61K 36/534; A61P 1/14

(52) U.S. Cl. CPC A61K 9/2077; A61K 36/23; A61K 36/534; A61P 1/14

(57) ABSTRACT

Recent studies in patients with irritable bowel syndrome, indigestion and constipation have shown the stomach to be a major site of disturbance in these disorders. For centuries herbs and their essential oil(s) have been used for relief of symptoms of various gastrointestinal disorders. There is currently no available technology that allows release of an essential oil(s) in the stomach without causing heartburn. In this patent multiparticulate compositions are disclosed which allow targeted and sustained delivery of essential oil(s) in the stomach and the intestines to treat GI disorders, without causing heartburn. These compositions will provide faster and broader symptom relief and prevent flare-ups of irritable bowel syndrome, indigestion and constipation. These compositions can also be used to treat other GI disorders such as gastroparesis, food intolerances, esophageal reflux, and inflammatory bowel diseases.

### FIELD OF THE INVENTION

The present invention is directed to controlled release formulations containing essential oil(s) which allow release in the stomach and the intestines, while avoiding release near the lower esophageal sphincter.

### BACKGROUND

The gastrointestinal (GI) system consists of the GI tract and associated organs. The GI tract consists of the oral cavity, pharynx, esophagus, stomach, small intestine, large intestine, and anal canal. The associated organs include the teeth, tongue, and glandular organs such as salivary glands, pancreas, gallbladder, and liver. The main functions of the GI system include ingestion and digestion of food, nutrient absorption, and excretion of waste products. The human digestive system is controlled by the neural network and hormones. Disturbance in any of these functions can lead to distressing symptoms.

GI symptoms such as abdominal pain, heartburn, abdominal discomfort, bloating, gas, diarrhea, constipation, nausea, and belching are bothersome, often recurrent and can impair quality of life. They can be triggered in healthy adults by food intake and psychological distress.

The proper opening and closing of the various sphincters that control the flow of the food from one segment of the GI tract to the next segment and the peristaltic contractions that move the food content in the lumen of the GI tract is done with smooth muscles. Improper coordination of the operation of the sphincters can cause heartburn, bloating, delayed gastric emptying. Improper peristaltic contractions can cause GI pain, discomfort, nausea, diarrhea, and constipation as well as other symptoms.

Until the 1980s the symptoms of irritable bowel syndrome (IBS) were called “Spastic Colon Syndrome” (SCS). The first peppermint oil product approved for SCS by UK's Medicines Control Agency during this time is called Colpermin®. This product contains peppermint oil (PO) dissolved in cooking oil and filled in an enteric-coated single-unit hard gelatin capsule). It is a delayed-release product designed to target the release of PO in the colon¹.

In the late 1980s the small intestine was identified as a location of disturbance and generation of symptoms observed with IBS². Further studies, in the early 2010s showed that the jejunum section of the small intestine showed cellular abnormalities in IBS³. IBS is called Abdominal Discomfort by lay persons.

A study reported in 2013⁴ showed for the first time that the stomach is the major site of origination of the GI symptoms in IBS. This study reported that the symptoms of IBS peaked at 30 to 60 minutes after a meal, when food was still in the stomach. The mechanism underlying the role of the stomach in generating symptoms of IBS was reported in 2023⁵. This study found that disturbances in the postprandial concentration of gut peptides (gastrin, insulin and ghrelin) in plasma may contribute to abnormal gastric function and consequently intestinal motility, which are manifested in the intensification of clinical symptoms, such as visceral hypersensitivity or irregular bowel movements in IBS patients.

IBS is classified as a functional GI disorder (i.e. a disorder where an organic cause for the disorder cannot be identified). There are two other functional disorders called Functional Dyspepsia (FD) and Functional Constipation (FC) which are also associated with disturbance in the stomach and their symptoms.

The importance of the stomach in generating symptoms of FD was reported initially in 2013⁶. This human study showed that the symptoms of this disorder originate in the stomach. FD is called Indigestion by lay persons.

Various recent publications have highlighted the importance of the stomach in most functional GI disorders^{7,8}.

The stomach has three main sections (cardia, fundus, and body). Cardia is the top part of the stomach that connects to the esophagus. Fundus is the rounded, bulbous section that is located above and to the left of the cardia. Body is the largest section where food is mixed and contracted. The small intestine has three sections (duodenum, jejunum, and ileum).

Various enteric coated drug delivery systems have been developed for managing GI disorders. In the case of Gastro-Esophageal Reflux Disease (GERD), proton pump inhibitors (PPIs) are delivered orally as enteric-coated multiparticulates to prevent release of the PPI in the stomach. The PPIs degrade quickly if they are exposed to the low pH in the stomach and need to be in blood circulation prior to their pharmacological action. This technology is highlighted in U.S. Pat. No. 7,544,370 B2 “Pantoprazole Multiparticulate Formulations” (used in the Protonix® commercial product). Aspirin is enteric coated to prevent stomach ulcers and bleeding.

The stretching of the muscles in the stomach (upper GI) is known to result in a bowel movement (lower GI) by the gastrocolic reflex. Recently, various drug delivery systems (enteric coated, non-enteric coated, rapid release in the stomach) have been evaluated to manage constipation in patients taking opioids for chronic pain. The evaluation of the various drug delivery systems for the delivery of methylnaltrexone to promote laxation is shown in U.S. Pat. No. 8,524,276 B2 “Oral Formulations and Lipophilic Salts of Methylnaltrexone” (used in the Relistor® commercial product). This work showed that delivery to the stomach was most important for addressing constipation, even though the peristaltic action in the large intestine (colon) results in laxation. This is because the smooth muscles in the large intestine are controlled by the enteric nervous system through the gastrocolic response.

Herbals and their extracts, such as essential oils, have been used for centuries to manage various distressing GI symptoms. The oral delivery of essential oils can be challenging as they are highly volatile and hydrophobic (oil soluble), and therefore difficult to formulate or to apply heat, especially during processing for targeted or controlled release delivery. However, the terpenes and terpenoids which are the primary ingredients of essential oils are stable in gastric pH conditions and their uncontrolled delivery in the stomach is known to result in heartburn.

In order to not cause the very distressing symptom of heartburn, which is often confused with having a heart attack, modern essential oil delivery systems have used enteric-coating technology. One of the more recently introduced delivery system for essential oils is the enteric-coated multiparticulates system containing peppermint oil, highlighted in U.S. Pat. No. 8,808,736 B2 “Enteric coated multiparticulate controlled release peppermint oil composition and related methods” (used in the IBgard® commercial product for IBS). This product was developed prior to 2013 when the small intestine was considered the site of disturbance in IBS. Similarly, the product FDgard® for FD was developed prior to 2013. Both of these products release their essential oil(s) in the small intestine, with minimal release in the stomach, as the small intestine was considered the main site of disturbance when they were developed.

The following U.S. Pat. Nos. 7,544,370 B2, 8,524,276 B2, and 8,808,736 B2 are incorporated by reference, as if set forth herein in their entirety.

Most essential oils have smooth muscle relaxing activity. This activity is highly desirable to relax the smooth muscles in the lining of the Body of the stomach, the pyloric sphincter, and the lining of the small intestine. However, this same activity is highly undesirable in the smooth muscles of the lower esophageal sphincter (in the Cardia section of the stomach) as the relaxation of this sphincter can cause back flow of stomach acids into the esophagus and trigger heartburn. The most distressing aspect of this symptom is that it is difficult to differentiate between heartburn and having a heart attack.

In order to reduce the incidence of heartburn symptoms, essential oils are dissolved in a cooking oil and administered as enteric-coated single-unit capsules or are prepared as enteric-coated multiparticulates filled in a capsule. The application of enteric-coating technology for delivery of essential oils is designed to prevent release of the essential oil(s) in the stomach. One major drawback with this approach is that essential oils would be more effective in managing GI symptoms if they acted in the stomach where they could address symptoms caused by smooth muscles in the lining of the stomach and the pylorus as well as delivering their well-known anti-inflammatory, anti-infective and pain relief activity of the oils.

Currently, there is no essential oils delivery system (for peppermint oil at a 180 mg therapeutically relevant single dose for IBS or 90 mg peppermint oil plus 50 mg caraway oil at a therapeutically relevant single dose for FD), that has been developed or commercialized to target controlled delivery in the stomach or stomach and the intestines.

The currently available delivery systems that deliver 180 mg of peppermint oil to treat IBS or 90 mg peppermint oil plus 50 mg caraway oil to treat FD use enteric coated single-unit capsules filled with essential oil(s) or enteric coated multiparticulates filled in a capsule. Both of these delivery systems have a major limitation as they are formulated to not release the essential oil(s) in the stomach. These systems were developed before the important role of stomach in generating symptoms of IBS, FD and FC was known and to avoid heartburn.

The other limitations of the 2 primary commercial technologies for essential oil(s) delivery are highlighted below:

In the Case of Enteric-coated Single-unit Capsules:
1. The oil-filled capsules are less dense than water and float in the stomach rather than going quickly through the stomach.
2. These capsules are dependent on gastric emptying to go from the stomach to the intestines which may not occur for several hours after consumption of the capsule. During this extended time the gelatin shell can hydrate, swell, and rupture the enteric coating resulting in dumping of the essential oil. This bolus of essential oil dissolved in cooking oil can float above the stomach fluid and cause the relaxation of the esophageal sphincter. The opening of this sphincter allows stomach acid to flow into the esophagus and results in the burning sensation in the chest called heartburn.
3. Even if these capsules empty the stomach without rupturing, their transit time through the intestines can be highly variable and their contents will dump over a small segment of the GI tract rather than allowing the essential oil to provide broad brush local exposure to the desired region of the GI tract.
4. These capsules have been shown to pass through the small intestine intact without releasing their essential oil(s)¹.

In the Case of Enteric-coated Multiparticulates:
1. The process by which these multiparticulates are prepared uses water to form the non-coated multiparticulates. When this water is removed, prior to coating, it results in the formation of air-filled pores throughout the multiparticulates. The coating(s) applied on top of the surface of the multiparticulates traps the air in the multiparticulates and makes them buoyant and sink slowly in the stomach fluid. A few of the enteric-coated multiparticulates have been observed to float above the simulated stomach fluid (0.1 M HCl solution) rather than sinking. Floating or slow sinking of the multiparticulates near the Cardia and Fundus (upper) sections of the stomach increases the risk of heartburn due to the possibility of leakage of essential oils and their exposure to the lower esophageal sphincter.
2. The enteric coating on these multiparticulates prevents release of any part of the active(s) in the stomach or the pyloric sphincter at the bottom of the stomach because the contents of these area have a pH (˜1.0 pH) that is below the pH (˜5.0 pH), at which the enteric-coating polymer used in these formulations can dissolve to release the active(s).
3. The enteric coating on the multiparticulates starts to dissolve after they have entered the duodenum where the contents have a pH which is closer to the minimum pH required to dissolve the enteric polymers. However, the residence time of the multiparticulates in the duodenum is short as it constitutes the shortest section (approximately 9 inches) of the small intestine. After the enteric coating dissolves, the subcoat (made up of gelatin) which separates the multiparticulates from the enteric coating must also dissolve prior to release of the active(s) in the lumen of the GI tract. This results in minimal exposure of the essential oil active(s) to the enteric receptors in the duodenum segment of the small intestine. The receptors in the stomach and the duodenum are known to be of great importance in managing undesirable GI symptoms, such as indigestion (FD). The global gastroenterologist community recognizes FD as a gastro-duodenal disorder.

An ideal system for delivery of essential oils to treat GI disorders that involve the stomach would be a system that allows:
1. Minimal release of the essential oil(s) in the Cardia section of the stomach (i.e. near the lower esophageal sphincter), to reduce the risk of heartburn.
2. Predictable passage through the pylorus even when it is in a closed position. Therefore, the system must contain particles with an average diameter of less than 3 millimeters, which can pass through the pylorus in its closed position.
3. Rapid hydration and sinking of particles away from the esophageal sphincter of the stomach and quick flow to the lower regions as well as a long residence time in the bottom section of the Body of the stomach.
4. Controlled release of between 25% to 100% of the essential oil(s) in the stomach to address the symptoms originating in the stomach and help with gastric emptying through the pyloric sphincter.
5. Release the remaining essential oil(s) within the targeted section of the small intestine (duodenum, duodenum plus jejunum or all of the small intestine segments) in a sustained manner.

The delivery system with the above characteristics needs be a modified release multiparticulate system with particles that are physically and chemically stable, significantly denser than stomach fluid, and rigid enough to withstand wetting with the acidic stomach fluids and the shear created by the peristaltic movements in the stomach. They must also be able to flow through a conical hopper without agglomerating during capsule filling or other downstream processing.

A critical advantage of using multiparticulates that are denser than the stomach fluid is that they allow for gastroretentive delivery⁹ of the essential oils from the bottom regions of the stomach. This allows for sustained release in the stomach and thereby provides longer exposure of the active essential oils to the receptors and muscles in the stomach.

When the multiparticulate system, used in IBgard® and FDgard®, was being developed for delivery of essential oils the important role of the stomach as the site of origination of the GI symptoms seen in Functional Dyspepsia (indigestion), IBS (abdominal discomfort) and Constipation was not known. Also, at this time there was no viable technology that could provide the ideal delivery characteristics mentioned above. Therefore, until recently most of the development effort was focused on modifying the enteric-coated delivery system with minimal release in the stomach.

The new insight regarding the important role of the stomach in generating symptoms of IBS, FD and FC led to an evaluation of various delivery technologies that may be able to safely deliver in the stomach and address their unmet medical needs.

The investigation and related experimentation resulted in several surprising findings which made it possible to develop a multiparticulate system with the desired properties.

It is a surprising finding that the small pores of microcrystalline cellulose (MCC)¹⁰ fiber bundles can be filled with essential oil(s) to a greater extent with longer high shear mixing times to allow longer release times in the intestines, while the larger pores allow for quicker release in the stomach.

It is a surprising finding that increasing the level of the methylcellulose binder can densify the resulting multiparticulates so that they sink faster in simulated gastric fluid, despite a relatively high content of 30% essential oils (these oils are lighter than gastric fluid and float on its surface) by weight in the multiparticulates.

It is a surprising finding that increasing the level of the methylcellulose binder prevents breakage of the multiparticulates and premature release of the essential oil(s) when these multiparticulates are wetted with simulated gastric fluid.

It is a surprising finding that the capsules filled with multiparticulates dried to a residual moisture level between 3.5% and 7.5% are room temperature stable during long term storage.

### SUMMARY

In one embodiment of the present invention, is a multiparticulates formulation containing essential oil(s) with controlled release in the stomach or stomach and intestines.

In another embodiment of the present invention, is a multiparticulates formulation containing essential oil(s) that allows ease of movement of stomach contents, through the pyloric sphincter, into the intestines without promoting movement of the stomach contents into the esophagus.

In another embodiment of the present invention, is a multiparticulates formulation containing essential oil(s) with controlled release in the stomach and intestines to manage undesirable gastrointestinal symptoms.

In another embodiment of the present invention, is a multiparticulates formulation containing essential oil(s) with controlled release in the stomach and intestines where the average diameter of the multiparticulates is less than 3 millimeters.

In one embodiment of the present invention, is a multiparticulates formulation containing essential oil(s) with controlled release in the stomach and intestines which is filled in a capsule.

In one embodiment of the present invention is a method of treatment for various GI disorders or to manage the symptoms of GI disorders.

In one embodiment of the present invention is a method of treatment for functional GI disorders or to manage the symptoms of functional GI disorders.

In one embodiment of the present invention is a method of treatment for functional GI disorders such as abdominal discomfort (IBS) or to manage the symptoms of abdominal discomfort (IBS).

In one embodiment of the present invention is a method of treatment for functional GI disorders such as upset stomach and indigestion (FD) or to manage the symptoms of upset stomach and indigestion.

In one embodiment of the present invention is a method of treatment for functional constipation (FC) or to manage the symptoms of constipation.

In one embodiment of the present invention is a method of treatment for various GI disorders or to manage the symptoms of GI disorders related to delayed gastric emptying or gastroparesis.

In one embodiment of the present invention is a method of treatment for food intolerances or food allergies and to manage their symptoms.

### BRIEF DESCRIPTION OF THE DRAWINGS

There are 4 drawings provided in the attached pdf file. They are:

FIG. 1. Anatomy of the Stomach

FIG. 1 shows the esophageal sphincter which controls the flow of food from the esophagus to the stomach and the pyloric sphincter that controls the flow of chyme from the stomach to the duodenum.

It also highlights the fundus part of the stomach which is used to store gas produced during digestion. The gas/liquid interface is where multiparticulates lighter than gastric fluid and enteric-coated oil filled capsules can float.

FIG. 2 Manufacturing Flow Chart

FIG. 2 presents a Manufacturing Flow Chart that describes the equipment and steps in the process for preparing the multiparticulates, encapsulation and packaging.

It shows that initially powders of MCC and MC along with optionally active(s) in a powder form are blended in the granulator bowl with mixing.

This is followed by the addition of essential oil(s) and water. The granulation is continued until the granulation endpoint is achieved.

The granules are moved from the granulator to the hopper of the extruder. The extrudates are formed and chopped into cylinders with a diameter similar to their length.

The cylinders are converted into wet spheres by the rotating force of the spheronizer drum.

The wet spheres are dried in the fluid bed dryer at around room temperature to a low moisture level (between 3.5% and 7.5%). The over size and undersize spheres are removed by screening.

The dried multiparticulates are then filled in capsules to the desired level of the active(s).

FIG. 3. A Multiparticulate with Pores (with cross-section for illustration)

FIG. 3 shows the 3 types of pores in an individual sphere-shaped multiparticulate. It illustrates, in the magnified cross-section of the particle, the micron range diameter pores (small pores less than 10 μm and large pores greater than 10 μm) within the MCC¹⁰, wherein the essential oil(s) are absorbed.

It also shows the largest pores prepared by the binding of the MCC particles with MC plus water, which are filled with air after the water added during granulation is removed during the drying step.

The multiparticulates may be milled into a coarse powder and formulated into a rapidly disintegration tablet.

FIG. 4. Simulated Release Profile in Water With Ph 7.0

FIG. 4 illustrates a release profile for the active(s) from the multiparticulates in a dissolution bath with slow mixing.

This simulation is focused on delivery to the stomach and the small intestine (duodenum, jejunum, and ileum).

The release in the first hour of 50% active reflects the transit time for the multiparticulates in the stomach.

The rest of the active (50%) release in the next 7.5 hours, which is the normal transit time in the small intestine for healthy adults and children.

### DETAILED DESCRIPTION

This disclosure is to convey preferred embodiments of the invention to those skilled in the art.

Essential oils are primarily composed of mixtures of terpenes or terpenoids which are highly volatile and relatively insoluble in water (hydrophobic). Therefore, they cannot be processed at high temperatures or incorporated in the typical aqueous based processes used to formulate most active ingredients.

MCC is a common excipient used in tablet formulations and is one of the few processing aids for formulation of multiparticulates prepared by extrusion spheronization. It has been used to prepare enteric-coated multiparticulates containing peppermint oil, highlighted in U.S. Pat. No. 8,808,736 B2 “Enteric coated multiparticulate controlled release peppermint oil composition and related methods” (used in the IBgard commercial product).

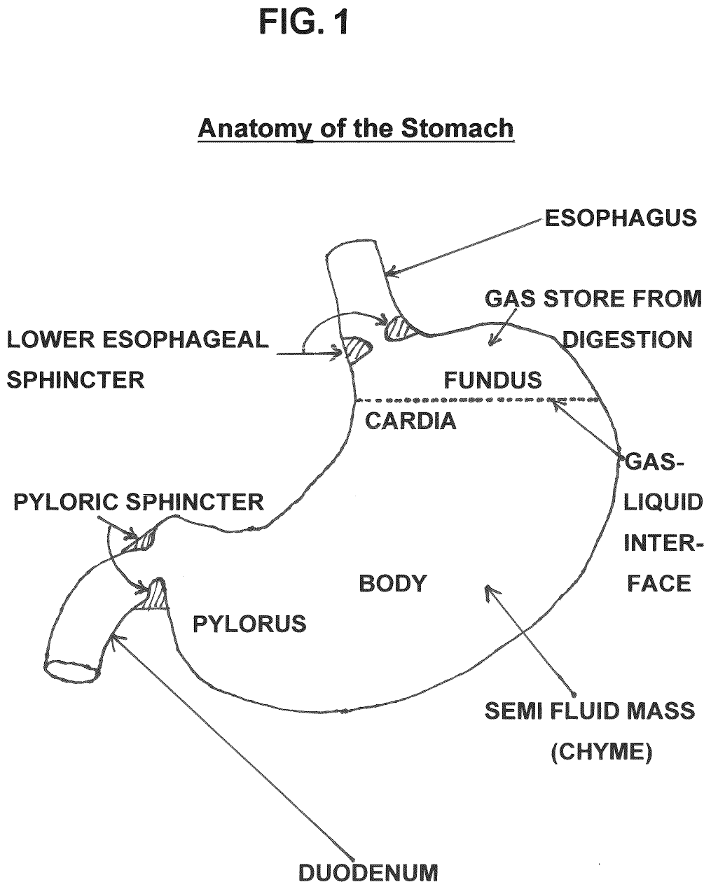

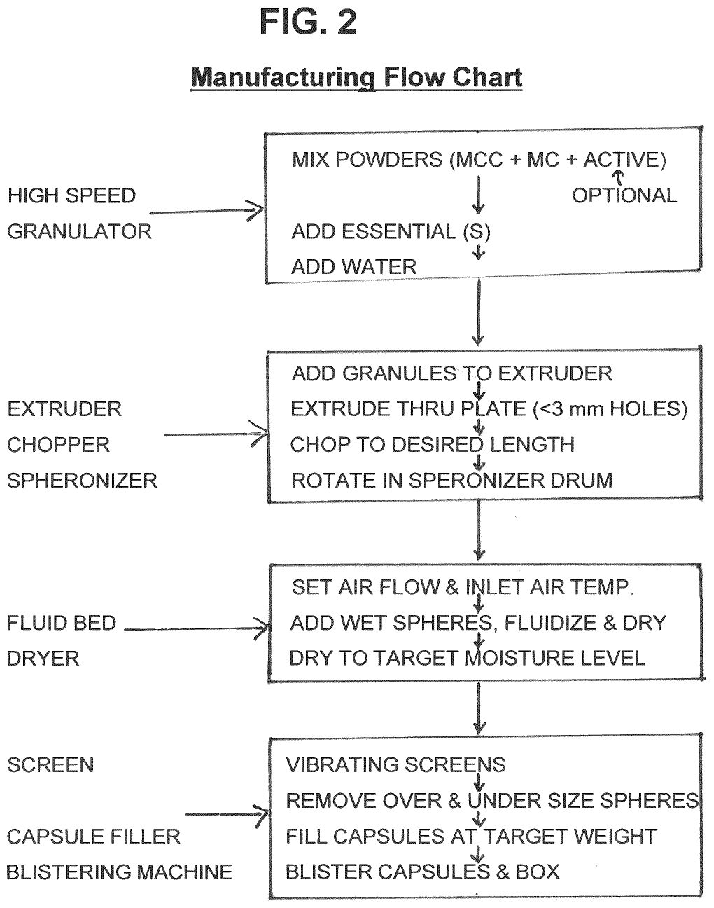

FIG. 1 (Anatomy of the Stomach) illustrates the 4 different sections of the stomach (Cardia, Fundus, Body, and Pylorus). The food and/or capsule (containing the multiparticulates described in the invention) passes the lower esophageal sphincter into the stomach. The capsule disintegrates and releases its contents of multiparticulates in the upper section (Cardia and Fundus) section of the stomach. The multiparticulates are wetted by the gastric fluid and the larger pores are filled with this fluid. The displacement of the air from these pores with gastric fluid as well as the smaller pores filled with essential oil(s) increases the density of the multiparticulates and allows them to sink and thereby move readily into the Body of the stomach. The sustained release feature of the formulation allows a minimal fraction of the essential oil(s) present in the multiparticulates to be released in the Cardia/Fundus region as they go into the Body of the stomach.

The multiparticulates described in this invention are designed to carry an essential oil or a combination of essential oils to the stomach and intestines. The multiparticulates are preferably spherical in shape and have an average diameter less than 3 mm (millimeters). Preferably, the average diameter of the multiparticulates is in the range of 0.5 to 2.0 mm.

Particles less than 3 mm in diameter can flow readily through the closed pyloric sphincter. This sphincter closes in the presence of food which allows digestion in the stomach. Therefore, the multiparticulates described in this invention are able to move from the stomach to the duodenum regardless of whether this sphincter (valve) is in a closed or open position. This provides a reliable release and exposure of the essential oil(s) in the stomach, the pylorus, and the intestines.

The multiparticulate may be prepared to contain one essential oil or a mixture of essential oils, including individual terpenes such as L-menthol. The essential oils and pure terpenes include but are not limited to peppermint oil, L-menthol, caraway oil, ginger oil, fennel oil, orange oil, turmeric oil, curcumin oil etc.

Ingredients that are oil soluble, such as curcuminoids, oil soluble vitamins etc., can be dissolved in the essential oil prior to incorporation in the MCC and subsequently prepared as multiparticulates. Ingredients that are available in powder form, such as curcumin powder, can be blended with the MCC and MC prior to preparation of the multiparticulates.

The process of preparation of multiparticulates requires the addition of a binder to the mixture of the essential oil and MCC. The function of the binder is to cross-link the particles of MCC with absorbed essential oil(s). This is illustrated in FIG. 3 (A multiparticulate with pores). The binding of the MCC particles with the binder requires the addition of the water to the mixture of MCC, essential oil and a binder. The water helps in forming a dough which can be extruded, chopped and spheronized. The binder may be cellulose-based, starch-based or povidone-based. Methylcellulose (MC) is the preferred binder in this formulation.

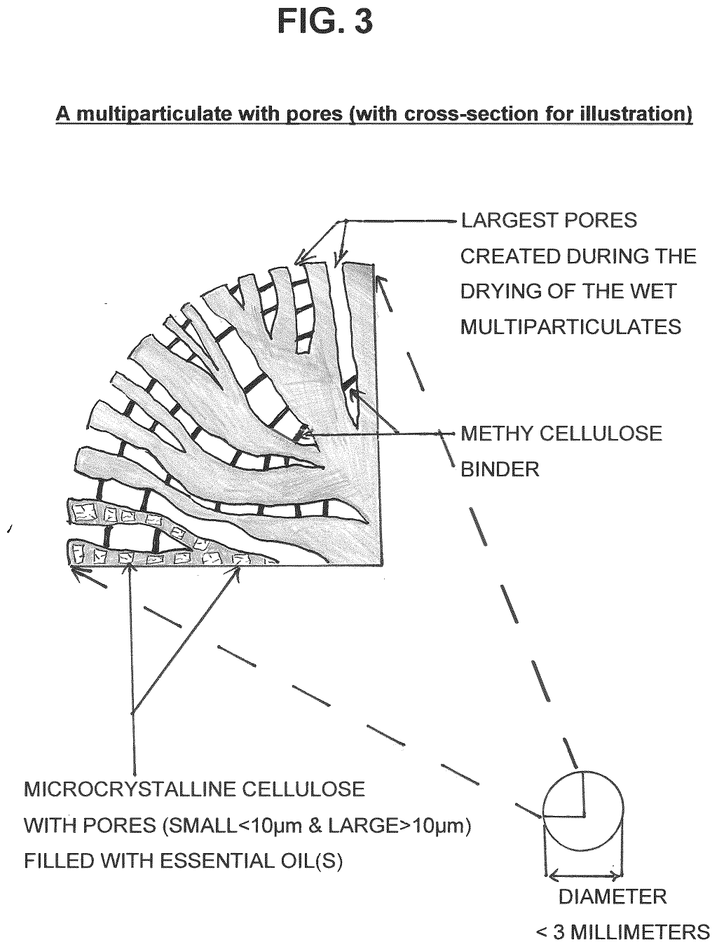

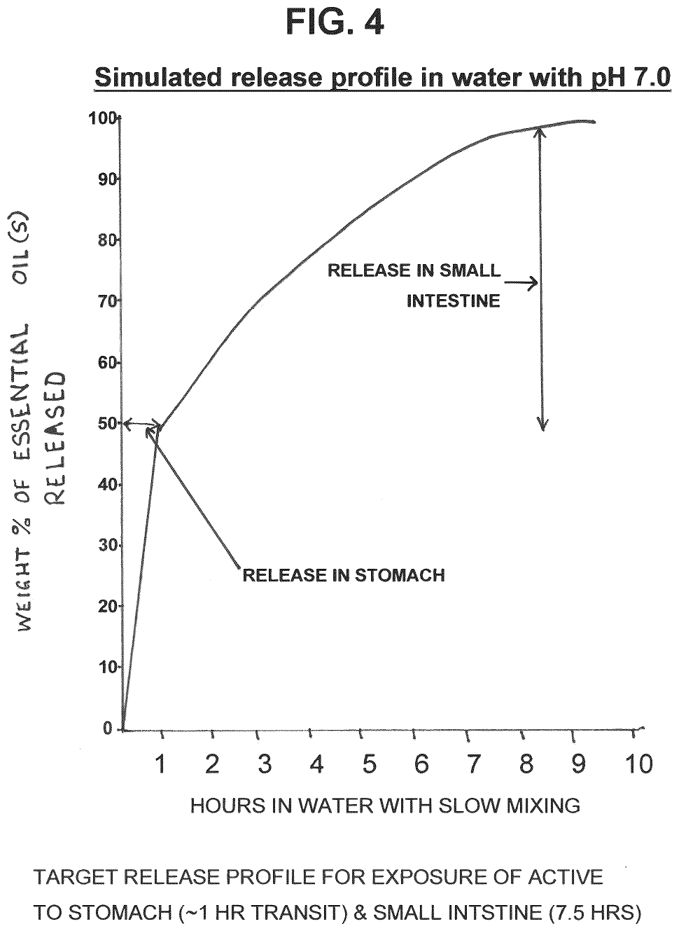

FIG. 2 (Manufacturing Flow Chart) describes the equipment and various steps in the manufacturing process, starting from the mixing of the various ingredients and finishing with the blistered capsules filled in a box.

The method for preparation of enteric-coated multiparticulates has been disclosed in the U.S. Pat. No. 8,808,736 B2. In this method for making the enteric-coated multiparticulates, individual cores are prepared as intermediate multiparticulates which are subsequently coated by three layers of coatings. Fluid bed coating is used to apply these different layers of coatings over the cores. These layers include a gelatin coat, a methacrylic co-polymer based enteric-coat and a finishing coat made of hydroxypropyl methyl cellulose. The cores are multiparticulates which contain an essential oil (such as peppermint oil), microcrystalline cellulose (MCC) and a binder (methylcellulose). These cores (i.e. intermediate multiparticulates) were developed to allow efficient coating with a gelatin coat. The optimum moisture content of dried cores prior to gelatin coating is in the range of 12 % to 18% (by KF moisture test), with a target level of 15 % moisture in these cores. This moisture level ensures that the essential oil(s) do not get removed from the multiparticulates during the long gelatin coating step.

The method for making the non-coated multiparticulates (cores) described in the above patent was used to prepare multiparticulates which were filled into hard gelatin capsules. Within 24 hours after filling the capsules it was noticed that all of the capsules were deformed. Different reasons for the deformation of capsule shells were evaluated. It was found that the deformation of the capsules was prevented when most of the unbound moisture was removed from the multiparticulates, by a second drying cycle, prior to filling the capsules. These were called re-dried multiparticulates, as the multiparticulates had initially been dried by a fluid bed process.

The processing conditions during the fluid bed drying step were modified to remove the un-bound moisture. This allowed production of multiparticulates with only one drying step which did not damage the capsules after they were filled and were room temperate stable.

The non-coated multiparticulates were evaluated by dropping 200 milligrams of the non-coated multiparticulates in a 200 Milliliter (ml) beaker (Cole-Parmer Boro 3.3) containing 150 ml of simulated gastric fluid [SGF] (0.1 M Hydrochloric Acid from Aldon Chemicals). The density of this SGF is similar to the density of gastric content⁹ (1.004 g/cubic centimeter), which is close to the density of water. When these non-coated multiparticulates were visually observed after they were dropped in the SGF, there was extensive surface erosion, breakage and premature release of the essential oils. The rapid release of the essential oil in SGF was visually observed as a very fine emulsion flowing out of the particles in the form of a mist.

This rapid and early release of essential oils such as peppermint oil in the upper part of the stomach (near the esophageal sphincter [valve]) is highly undesirable. When the essential oils such as peppermint oil are released rapidly, they form large droplets which float and come in contact with the esophageal valve. The muscle relaxing activity of peppermint oil opens this valve resulting in the back flow of the acidic gastric fluid into the esophagus, leading to the distressing symptom of heartburn.

Various formulas and process conditions were evaluated for production of the non-coated multiparticulates with a) lower attrition of multiparticulates when exposed to SGF, b) with higher sinking rates in SGF and c) with slow and sustained release of the essential oil active(s) in SGF.

During testing of various processing conditions, it has been found that increasing the high shear mixing time, when the essential oil is mixed with the MCC in the granulator, slows down the rapid release from the multiparticulates, in SGF (the extra energy input due to the longer mixing may allow the essential oil to fill more of the small pores (<10 μm diameter) of MCC¹⁰.

After testing various formulas, it has been found that increasing the methylcellulose level from approximately 5.26 % to at least 6.00 % (w of MC/w % MCC) resulted in multiparticulates with higher density (faster sinking in SGF) and greater strength (less attrition when exposed to SGF.

FIG. 4 (Simulated release profile in water with pH 7.0) is an illustration of a release that would be anticipated to allow controlled exposure of the active(s) in essential oil(s) to the stomach and all 3 regions of the small intestine. One of the advantages of this invention is that it reduces the risk of causing heartburn due to 2 main reasons.
1. Multiparticulates hydrate, sink and flow away from esophageal sphincter quickly because of their higher density (when practically all of the largest pores in the multiparticulates get filled with chyme, displacing the air in these pores). The micron size pores are filled with essential oil(s).
2. The slow (sustained) release of essential oil(s) from the multiparticulates only allows a small fraction of the active to be released in the upper part of stomach, while they are sinking, which limits exposure of actives (smooth muscle relaxers) to the smooth muscles of the lower esophageal sphincter.

FIG. 4 shows a release profile that is ideal for a functional disorder like irritable bowel syndrome (IBS), because this disorder involves symptoms centered around the stomach (bowel movement and bloating) and the length of the small intestine (GI pain and discomfort). Upset stomach and indigestion (FD) is known to involve the stomach and the duodenum, therefore the multiparticulates for this disorder require a faster delivery than the one for abdominal discomfort (IBS).

The delivery of essential oils (terpenes and terpenoids) in the stomach and small intestine, without a significant risk of heartburn has not been evaluated for various unmet medical needs. This invention with a selected essential oil or a combination of essential oils which is/are appropriately absorbed in MCC pores along with an appropriate level of binder will be effective in treating the symptoms associated with IBS, FD and FC.

This invention may also treat slow gastric emptying or gastroparesis by releasing essential oil(s) with smooth muscle relaxing activity inside the pyloric sphincter to open the closed sphincter and allow gastric contents to flow from the stomach into the intestines.

The controlled or sustained release of essential oils in the stomach will expose the actives to the lining and receptors of the stomach and trigger the gastrocolic reflex. This will promote laxation and relieve the symptoms of constipation and bloating.

The controlled or sustained release of essential oils in the stomach and stomach plus the intestines will expose the actives to their lining and receptors and provide their anti-inflammatory, antinociceptive, muscle relaxing properties to attenuate food intolerances and food allergies.

The multiparticulates described above can be administered orally through the use of a conventional dosage form such as a capsule.

The tablet or powder blend to manage constipation may be formulated, after milling (with a grinder, CO-Mill, Fitz-Mill etc.) of the multiparticulates, and blending with excipients such as lecithin (surfactant), carbonate or bicarbonate salt (for carbon dioxide gas production). Other tableting excipients can be blended along with the super disintegrants (such as croscarmellose sodium), before compressing the mixture into a tablet.

### EXAMPLES

This section provides specific examples of the multiparticulate composition of this invention. These examples are provided to highlight certain preferred embodiments of the invention, but the scope of the invention is not limited to the examples shown in this section.

### Example 1

### Preparation of a Multiparticulate Composition for IBS (Abdominal Discomfort)

Added 33.25 kg MCC (PH102), and 2.00 kg methylcellulose (A15LV) to the high shear granulator. Mixed the powders until a uniform blend was prepared.

Then added 11.67 kg of distilled peppermint oil to the powder blend with a high shear mixing for 8 minutes. Then added water until proper granules were produced.

The granulated wet mass was extruded, chopped, and spheronized. The spheronized pellets were dried to a moisture level of 5.5% by KF in a fluid bed dryer. The dried multiparticulates (pellets) were filled in individual size 00 capsules, with each capsules filled to contain about 90 mg of peppermint oil [equivalent to half of the dose of this essential oil used in the clinically tested single-unit enteric coated commercial capsules of Colpermin®]

### Example 2

### Preparation of a Multiparticulate Composition for FD (Upset Stomach/Indigestion)

Added 33.25 kg MCC (PH102), and 2.00 kg methylcellulose (A15LV) to the high shear granulator. Mixed the powders until a uniform blend was prepared. Then added 7.5 kg of distilled peppermint oil and 4.17 kg of distilled caraway oil to the powder blend with high shear mixing for 7.5 minutes. Added water until proper granules (wet mass) were produced.

The granulated wet mass was extruded, chopped, and spheronized. The spheronized pellets were dried in a fluid bed dryer to residual moisture (by KF) to a moisture level of 5.5%. The dried multiparticulates (pellets) were filled in individual capsules, with each capsules filled to contain about 45 mg of peppermint oil and about 25 mg of caraway oil [equivalent to half of the dose of these essential oils used in the clinically tested single-unit enteric-coated commercial capsules of Menthacarin®].

### Example 3

### Preparation of a Multiparticulate Composition in a Tablet for Constipation

Added 33.25 kg MCC (PH102), and 2.00 kg methylcellulose (A15LV) to the high shear granulator. Mixed the powders until a uniform blend was prepared. Then add 11.67 kg of distilled peppermint oil to the powder blend with high shear mixing for 8 minutes. Added water until proper granules (wet mass) were produced.

The granulated wet mass was then extruded, chopped, and spheronized. Then dried spheronized pellets to a level of 5.5% (KF) in a fluid bed dryer. Then milled (Fitz Mill) the dried multiparticulates (pellets) to make a coarse powder. Blended (Conical Blender) the coarse powder with MCC (about 30% w/w of blend), 3.33 kg Curcumin Powder (95% curcuminoids extracted from Turmeric), lecithin powder (about 7% w/w of blend), Sodium Bicarbonate (about 3% w/w of blend) and the super-disintegrant croscarmellose sodium (about 10% w/w of blend). Compressed the blend into tablets. Used SGF to confirm a disintegration time in SGF testing of less than 10 minutes.

### Example 4

### Preparation of Multiparticulates With Fast Dissolving Coating

Coated the dried spheronized multiparticulates described in Example 1 in a fluid bed coater to a 1% w/w level (weight gain by the multiparticulates). Used a 10% solution of low molecular weight hydroxypropyl methylcellulose (HPMC, Methocel E15LV) in water as the coating solution. Then filled the coated multiparticulates in hard gelatin capsules.

1. Non-enteric coated multiparticulate compositions containing essential oil(s), microcrystalline cellulose and a binder with controlled release in the stomach or stomach and intestines.

2. The multiparticulate compositions formulation of claim 1, where the binder is methyl cellulose.

3. The multiparticulate compositions formulation of claim 1, where the binder's weight percentage of methyl cellulose as compared to microcrystalline cellulose is 6% or greater.

4. The multiparticulate compositions of claim 1, where the level of binder is effective in making the multiparticulate compositions denser than the stomach fluid and is effective in reducing excessive erosion of the multiparticulates in SGF.

5. The multiparticulate compositions of claim 1, where the average diameter of the multiparticulates is less than 3 millimeters.

6. The multiparticulate compositions of claim 1, where the multiparticulates are dried to a moisture level of less than 8% and filled in a capsule.

7. The multiparticulate compositions of claim 1, which are milled into a powder.

8. The multiparticulate compositions of claim 1, which is milled into a powder and then blended with lecithin powder plus a bicarbonate salt, followed by filling the blend into a capsule.

9. The multiparticulate compositions of claim 1, which is milled into a powder and blended with lecithin powder, a bicarbonate salt and croscarmellose sodium plus tableting aids (such as MCC, magnesium stearate, and starch) and compressed into a tablet.

10. The multiparticulate compositions of claim 1, which releases greater than 25% of the essential oil(s) within one hour in SGF and the remaining 75% in the next 23 hours.

11. The multiparticulate compositions of claim 1, which releases greater than 75% of the essential oil(s) within one hour in SGF and the remaining oil(s) in the next 23 hours.

12. The multiparticulate compositions of claim 1, that allow controlled delivery of essential oils in the stomach while lowering the potential for heartburn by minimizing release of the essential oils near the esophagus.

13. A method of treatment for various GI disorders or the symptoms of GI disorders by using a multiparticulates formulation containing an essential oil, microcrystalline cellulose and a binder with controlled release in the stomach and intestines.

14. The method of claim 12, where the GI disorder is a functional GI disorder.

15. The method according to claim 12 wherein said GI disorder is selected from a group consisting of upset stomach, indigestion (dyspepsia), abdominal discomfort (irritable bowel syndrome), constipation, and delayed gastric emptying (gastroparesis).

16. The method according to claim 12 wherein the GI disorder in due to food intolerance or food allergies.
